(19) United States
(12) Patent Application Publication
(10) Pub. No.: US 2013/0171385 A1
(43) Pub. Date: Jul. 4, 2013

(54) REACTING CYCLOPENTADIENE AND MALEIC ANHYDRIDE FOR THE PRODUCTION OF PLASTICIZERS

(71) Applicant: EXXONMOBIL RESEARCH AND ENGINEERING COMPANY, Annandale, NJ (US)

(72) Inventors: Jihad Mohammed Dakka, Whitehouse Station, NJ (US); Stephen Zushma, Clinton, NJ (US); Edmund John Mozeleski, Califon, NJ (US); Diana S. Smirnova, High Bridge, NJ (US); Lisa Saunders Baugh, Ringoes, NJ (US); Allen David Godwin, Seabrook, TX (US)

(73) Assignee: EXXONMOBIL RESEARCH AND ENGINEERING COMPANY, Annandale, NJ (US)

(21) Appl. No.: 13/723,513

(22) Filed: Dec. 21, 2012

Related U.S. Application Data
(60) Provisional application No. 61/581,230, filed on Dec. 29, 2011.

(51) Int. Cl. C07C 67/08; C07C 69/753; C08K 5/12

(52) U.S. Cl. CPC C07C 67/08; C08K 5/12; C07C 69/753
USPC 428/35.2; 524/285; 523/122; 521/145; 560/120; 560/114; 428/36.9; 442/104

(57) ABSTRACT

Multi-esters of the formula:

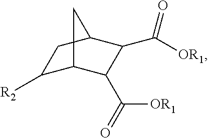

wherein each R₁ is the hydrocarbon residue of a C₄ to C₁₃ OXO-alcohol averaging from 0.2 to 5.0 branches per residue, and R₂ is hydrogen or an ester group, processes of making the multi-esters, use of the multi-esters as plasticizers, polymer compositions containing such plasticizers, and articles containing such plasticizers.

### CROSS-REFERENCE TO RELATED APPLICATION

This application claims the benefit of U.S. Provisional Application Ser. No. 61/581,230, filed on Dec. 29, 2011; which is incorporated herein in its entirety by reference.

### FIELD

This disclosure is related to a reaction route to non-phthalate, aromatic OXO multi-ester plasticizers for polymer compositions.

### BACKGROUND

Plasticizers are incorporated into a resin (usually a plastic or elastomer) to increase the flexibility, workability, or distensibility of the resin. The largest use of plasticizers is in the production of “plasticized” or flexible polyvinyl chloride (PVC) products. Typical uses of plasticized PVC include films, sheets, tubing, coated fabrics, wire and cable insulation and jacketing, toys, flooring materials such as vinyl sheet flooring or vinyl floor tiles, adhesives, sealants, inks, and medical products such as blood bags and tubing, and the like.

Other polymer systems that use small amounts of plasticizers include polyvinyl butyral, acrylic polymers, nylon, polyolefins, polyurethanes, and certain fluoroplastics. Plasticizers can also be used with rubber (although often these materials fall under the definition of extenders for rubber rather than plasticizers). A listing of the major plasticizers and their compatibilities with different polymer systems is provided in “Plasticizers,” A. D. Godwin, in Applied Polymer Science 21st Century, edited by C. D. Craver and C. E. Carraher, Elsevier (2000); pp. 157-175.

Plasticizers can be characterized on the basis of their chemical structure. The most important chemical class of plasticizers is phthalic acid esters, which accounted for 85% worldwide of PVC plasticizer usage in 2002. However, in the recent past there has been an effort to decrease the use of phthalate esters as plasticizers in PVC, particularly in end uses where the product contacts food, such as bottle cap liners and sealants, medical and food films, or for medical examination gloves, blood bags, and IV delivery systems, flexible tubing, or for toys, and the like. For these and most other uses of plasticized polymer systems, however, a successful substitute for phthalate esters has heretofore not materialized.

One such suggested substitute for phthalates are esters based on cyclohexanoic acid. In the late 1990's and early 2000's, various compositions based on cyclohexanoate, cyclohexanedioates, and cyclohexanepolyoate esters were said to be useful for a range of goods from semi-rigid to highly flexible materials. See, for instance, WO 99/32427, WO 2004/046078, WO 2003/029339, WO 2004/046078, U.S. Application No. 2006-0247461, and U.S. Pat. No. 7,297,738.

Other suggested substitutes include esters based on benzoic acid (see, for instance, U.S. Pat. No. 6,740,254, and also co-pending, commonly-assigned, U.S. Provisional Patent Application No. 61/040,480, filed Mar. 28, 2008 (and related EP Application No. EP08158375.9 filed on Jun. 17, 2008) and polyketones, such as described in U.S. Pat. No. 6,777,514; and also co-pending, commonly-assigned, U.S. Patent Publication No. 2008/0242895, filed Mar. 28, 2008. Epoxidized soybean oil, which has much longer alkyl groups (C₁₆ to C₁₈) has been tried as a plasticizer, but is generally used as a PVC stabilizer. Stabilizers are used in much lower concentrations than plasticizers. Co-pending and commonly-assigned U.S. Provisional Patent Application No. 61/203,626, filed Dec. 24, 2008 (and related U.S. Non-provisional application Ser. Nos. 12/653,744 filed on Dec. 17, 2009 and 12/971,629 filed Dec. 17, 2010), discloses triglycerides with a total carbon number of the triester groups between 20 and 25, produced by esterification of glycerol with a combination of acids derived from the hydroformylation and subsequent oxidation of C₃ to C₉ olefins, having excellent compatibility with a wide variety of resins.

U.S. Pat. No. 3,297,725 to Guendel et al. discloses stabilized highly polymerized plastic compositions, such as polyvinyl chloride, containing plasticizers which are obtained by epoxidizing esters of monobasic or polybasic unsaturated cycloaliphatic carboxylic acids with the aid of organic peroxy-acids or mixtures of hydrogen peroxide and organic acids, whereby an epoxide group is formed at the cycloaliphatic double bond. Alcohols useful to esterify the unsaturated cycloaliphatic acid include 2-ethylbutanol and 2-ethylhexanol, and those formed by catalytic addition of carbon monoxide and hydrogen to olefins with subsequent hydrogenation of the resulting hydrocarbons in accordance with the well-known oxo-process. The starting ester 3,6-endomethylene-Δ⁴-etrahydrophthalic acid-di-2-ethylbutyl ester is utilized to form 4,5-epoxy-3,6-endomethylene-hexahydrophthalic acid-di-2-ethylbutyl ester.

U.S. Pat. No. 2,963,490 to Rowland et al. discloses 4,5-epoxyhexahydrophthalates, which may be bridged at the 3-6 positions with methylene, wherein the ester portions are alkyl residues of saturated monohydric alcohols, including 2-ethylbutanol, 2-methylpentanol, 2-methylhexanol, 2-ethyhexanol, 7-methyloctanol, 2,6-dimethyl-4-heptanol, 3,3,5-trimethylhexanol, 3-isopropyl-5-methylhexanol, 3,7-dimethyloctanol, 9-methylnonanol, 15-ethylhexadecanol and the like. The unsaturated cycloaliphatic esters used as starting materials can be prepared by subjecting selected cyclic monounsaturated dicarboxylic acids or anhydrides to conventional esterification procedures by reacting the acid or the anhydride with at least one aliphatic monohydric alcohol in the presence of a catalyst. The cyclic monounsaturated dicarboxylic acids or corresponding anhydrides may be synthesized by a Diels-Alder addition reaction, such as where maleic anhydride is reacted with acyclic or alicyclic dienes, including 1,3-cyclopentadiene yielding nonbridged and bridged compounds, respectively.

U.S. Pat. No. 7,319,161 to Noe et al. discloses a process for preparing cyclohexanedicarboxylic acids or derivatives thereof, such as esters and/or anhydrides, encompassing the reaction of diene/maleic acid anhydride mixtures, in particular of butadiene/maleic acid anhydride mixtures or of mixtures of maleic acid anhydrides and C₅-dienes to give alkyl-substituted or unsubstituted cyclohexenedicarboxylic acid anhydrides in the condensed phase, ester formation, and then hydrogenation to give the corresponding cyclohexanedicarboxylic acid derivative, and also to the use of the cyclohexanedicarboxylic acids or derivatives thereof prepared according to the disclosure as plasticizers for plastics. The alcohols used to esterify the acid anhydrides may be linear or branched alcohols having 1-18 carbon atoms or oxoalcohols which are the hydroformulation products of C₅ to C₁₂ alkenes.

Matsuda et al., “Cyclohexane Carboxylates for use as Plasticizers”, Kogyo Kagaku Zasshi, vol. 62, pp. 1838-1841 (1959) discloses synthesis and testing of esters of 3,6-endo-methylenehexahydrophthalates as plasticizers, including 2-ethylhexanol di-esters and n-butanol di-esters.

JP63-057657 discloses tetracarboxylic acid esters as plasticizers, prepared for example from di(2-ethylhexyl)ester of 3,6-methylenetetrahydrophthalic acid.

JP06-306252 discloses compounding a cyclopolyolefin resin with a specific ester compound described by formulae (I)-(IV) thereof.

JP07-011074 discloses blending a polypropylene-based resin and/or a polyolefinic thermoplastic elastomer with a specific alicyclic dicarboxylic acid ester of formulae (I) or (II) thereof, wherein formula (II) can contain an endomethylene and can be esterified with 6-28C alkyl or alkenyl. The disclosure mentions the diisononyl ester of 3,6-methylenetetrahydrophthalic acid.

JP07-173342 discloses compounding a resin component consisting of a polypropylene-based resin and a polyolefinic thermoplastic elastomer with one or more alicyclic dicarboxylic acid esters of the formula (A), wherein the ester groups are 6-28C alkyl or alkenyl.

To date, none of the prior art compounds or compositions has demonstrated satisfactory equivalence to conventional phthalate plasticizers for use with PVC polymers.

Thus what is needed is a method of making a general purpose non-phthalate plasticizer having suitable melting or chemical and thermal stability, pour point, glass transition, increased compatibility, good performance and low temperature properties.

### SUMMARY

In one aspect, the present application is directed to multi-esters of the formula:

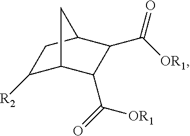

wherein each R₁ is the hydrocarbon residue of a C₄ to C₁₃ OXO-alcohol averaging from 0.2 to 5.0 branches per residue, and R₂ is hydrogen or an ester group.

Preferably, the multi-esters have hydrocarbon residues averaging from 0.05 to 0.4 branches per residue at the alcoholic beta carbon, and/or averaging at least 1.3 to 5.0 methyl branches per residue, more preferably from 0.35 to 1.5 pendant methyl branches per residue.

In one embodiment, the multi-esters are diesters, wherein R₂ is H.

Alternatively, the multi-esters are tri-esters of the formula:

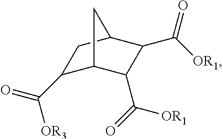

wherein R₁ and R₃ are hydrocarbon residues of the same or different OXO-alcohols, and preferably hydrocarbon residues of the same OXO-alcohol, having at least one pendant methyl branch per residue.

Another aspect of the present disclosure is directed to a process for making multi-esters comprising: reacting cyclopentadiene with maleic anhydride to form compounds of the following formula (I):

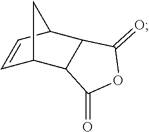 [SMILES: O=C1OC(=O)C2C3C=CC(C3)C12]

optionally conducting a first esterification step by contacting said compounds of formula (I) with at least one C₄ to C₁₃ OXO-alcohol of the formula R₁OH under esterification conditions to form compounds of the following formula (II):

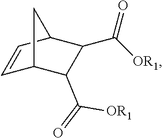

wherein R₁ is a hydrocarbon residue of the OXO-alcohol R₁OH; hydroformylating said compounds of formulae (I) or (II), and oxidizing said hydroformylated compounds to form multi-functional compounds of the following formula (IIa):

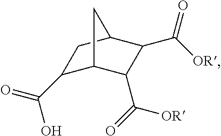

wherein R′ can be H or R₁; and reacting said multi-functional compounds of formula (IIa) with OXO-alcohols of the formula R₁OH if R′ is H, or if R′ is R₁ with an OXO-alcohol of the formula R₃OH, under esterification conditions to form tri-esters of the following formula:

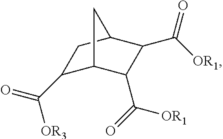

wherein each R₁ and R₃ are the same or different hydrocarbon residues of said C₄ to C₁₃ OXO-alcohols averaging from 0.2 to 5.0 branches per residue, such as wherein the hydrocarbon residue averages from 0.05 to 0.4 branches per residue at the alcoholic beta carbon, and/or wherein the hydrocarbon residue averages at least 1.3 to 5.0 methyl branches per residue, preferably from 0.35 to 1.5 pendant methyl branch per residue.

Preferably, the process is conducted such that each R₁ and R₃ are hydrocarbon residues of the same OXO-alcohol, having at least one pendant methyl branch per residue.

Alternatively, the disclosure is directed to a process for making multi-esters comprising: contacting maleic anhydride with at least one C₄ to C₃ OXO-alcohol of the formula R₁OH under esterification conditions to form compounds of the following formula (III):

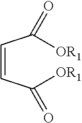

reacting the compounds of formula (III) with cyclopentadiene to form compounds of the following formula (II):

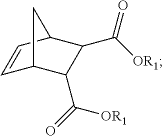

hydroformylating said compounds of formula (II), and oxidizing said hydroformylated compounds to form multi-functional compounds of the following formula (IIb):

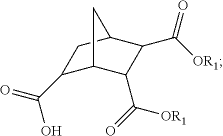

reacting said multi-functional compounds of formula (IIb) with an OXO-alcohol of the formula R₃OH, under esterification conditions to form tri-esters of the following formula:

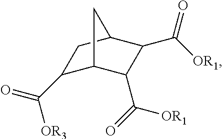

wherein each R₁ and R₃ are the same or different hydrocarbon residues of said C₄ to C₁₃ OXO-alcohols averaging from 0.2 to 5.0 branches per residue, such as wherein the hydrocarbon residue averages from 0.05 to 0.4 branches per residue at the alcoholic beta carbon, and/or wherein the hydrocarbon residue averages at least 1.3 to 5.0 methyl branches per residue, preferably from 0.35 to 1.5 pendant methyl branch per residue.

Advantageously, each of R₁ and R₃ are hydrocarbon residues of the same OXO-alcohol, having at least one pendant methyl branch per residue.

Alternatively, the disclosure is directed to another process for making multi-esters comprising: reacting cyclopentadiene with maleic anhydride to form compounds of the following formula (I):

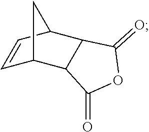 [SMILES: O=C1OC(=O)C2C3C=CC(C3)C12]

reacting the compounds of formula (I) with CO and OXO-alcohols of the formula R₁OH if R′ is H, or if R′ is R₁ with an OXO-alcohol of the formula R₃OH, in the presence of a metal catalyst to form compounds of the following formula (IV):

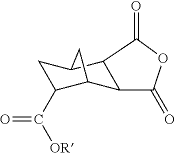

reacting the compounds of formula (IV) with OXO-alcohols of the formula R₁OH if R′ is H, or if R′ is R₁ with an OXO-alcohol of the formula R₃OH, under esterification conditions to form tri-esters of the following formula:

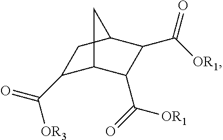

wherein each R₁ and R₃ are the same or different hydrocarbon residues of said C₄ to C₁₃ OXO-alcohols averaging from 0.2 to 5.0 branches per residue, such as wherein the hydrocarbon residue averages from 0.05 to 0.4 branches per residue at the alcoholic beta carbon, and/or wherein the hydrocarbon residue averages at least 1.3 to 5.0 methyl branches per residue, preferably from 0.35 to 1.5 pendant methyl branch per residue.

Advantageously, each of R₁ and R₃ are hydrocarbon residues of the same OXO-alcohol, having at least one pendant methyl branch per residue.

Alternatively, the disclosure is directed to still another process for making multi-esters comprising: reacting cyclopentadiene with maleic anhydride to form compounds of the following formula (I):

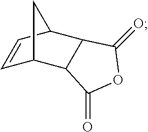 [SMILES: O=C1OC(=O)C2C3C=CC(C3)C12]

conducting a first esterification step by contacting said compounds of formula (I) with at least one C₄ to C₁₃ OXO-alcohol of the formula R₁OH under esterification conditions to form compounds of the following formula (II):

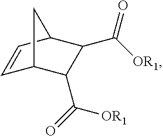

wherein R₁ is a hydrocarbon residue of the OXO-alcohol R₁OH; reacting the compounds of formula (II) with CO and OXO-alcohols of the formula R₁OH if R′ is H, or if R′ is R₁ with an OXO-alcohol of the formula R₃OH, in the presence of a metal catalyst to form tri-esters of the following formula:

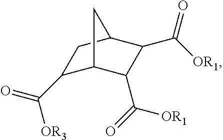

wherein each R₁ and R₃ are the same or different hydrocarbon residues of said C₄ to C₁₃ OXO-alcohols averaging from 0.2 to 5.0 branches per residue, such as wherein the hydrocarbon residue averages from 0.05 to 0.4 branches per residue at the alcoholic beta carbon, and/or wherein the hydrocarbon residue averages at least 1.3 to 5.0 methyl branches per residue, preferably from 0.35 to 1.5 pendant methyl branch per residue.

Advantageously, each of R₁ and R₃ are hydrocarbon residues of the same OXO-alcohol, having at least one pendant methyl branch per residue.

Alternatively, the disclosure is directed to still yet another process for making multi-esters comprising: contacting maleic anhydride with at least one C₄ to C₁₃ OXO-alcohol of the formula R₁OH under esterification conditions to form compounds of the following formula (III):

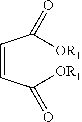

reacting the compounds of formula (III) with cyclopentadiene to form compounds of the following formula (II):

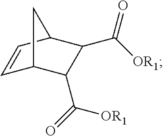

reacting the compounds of formula (II) with CO and OXO-alcohols of the formula R₁OH if R′ is H, or if R′ is R₁ with an OXO-alcohol of the formula R₃OH, in the presence of a metal catalyst to form tri-esters of the following formula:

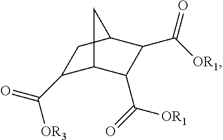

wherein each R₁ and R₃ are the same or different hydrocarbon residues of said C₄ to C₁₃ OXO-alcohols averaging from 0.2 to 5.0 branches per residue, such as wherein the hydrocarbon residue averages from 0.05 to 0.4 branches per residue at the alcoholic beta carbon, and/or wherein the hydrocarbon residue averages at least 1.3 to 5.0 methyl branches per residue, preferably from 0.35 to 1.5 pendant methyl branch per residue.

Advantageously, each of R₁ and R₃ are hydrocarbon residues of the same OXO-alcohol, having at least one pendant methyl branch per residue.

In a further embodiment, the disclosure is directed to a plasticizer comprising the multi-esters of the formula:

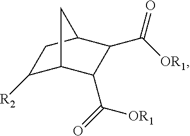

wherein each R₁ is the hydrocarbon residue of a C₄ to C₁₃ OXO-alcohol averaging from 0.2 to 5.0 branches per residue, and R₂ is hydrogen or an ester group.

In a further embodiment, the disclosure is directed to a polymer composition comprising a polymer and at least one plasticizer comprising multi-esters of the formula:

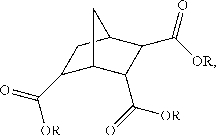

wherein each R is the hydrocarbon residue of a C₄ to C₁₃ OXO-alcohol averaging from 0.2 to 5.0 branches per residue, such as wherein the hydrocarbon residue averages from 0.05 to 0.4 branches per residue at the alcoholic beta carbon, and/or wherein the hydrocarbon residue averages at least 1.3 to 5.0 methyl branches per residue, preferably from 0.35 to 1.5 pendant methyl branch per residue.

Preferably the polymer of the composition is selected from the group consisting of vinyl chloride resins, polyesters, polyurethanes, ethylene-vinyl acetate copolymer, rubbers, poly(meth)acrylics and combinations thereof.

The present disclosure is still further directed to the use of such multi-esters as plasticizers for resin compositions, plastisols and articles to provide phthalate-free plasticizers, resin compositions, plastisols and articles.

These and other objects, features, and advantages will become apparent as reference is made to the following detailed description, embodiments, examples, and appended claims.

### DETAILED DESCRIPTION

All numerical values within the detailed description and the claims herein are modified by “about” or “approximately” the indicated value, and take into account experimental error and variations that would be expected by a person having ordinary skill in the art.

There is an increased interest in developing new plasticizers that are non-phthalates and which possess good plasticizer performance characteristics but are still competitive economically. The present disclosure is directed towards non-phthalate, multi-ester plasticizers, particularly OXO-ester plasticizers, that can be made from low cost feeds and employ fewer manufacturing steps in order to meet economic targets.

One route to non-phthalate plasticizers of the present disclosure is by reacting maleic anhydride and cyclopentadiene via Diels-Alder reaction to produce bifunctional compounds of the formula:

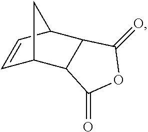 [SMILES: O=C1OC(=O)C2C3C=CC(C3)C12]

followed by either epoxidation or hydroformylation of the carbon-carbon double bond, oxidation and subsequent esterification of the resulting compounds. Alternatively, esterification of the bifunctional compound can precede either epoxidation or hydroformylation.

In a particularly advantageous embodiment, the following reaction scheme is used:

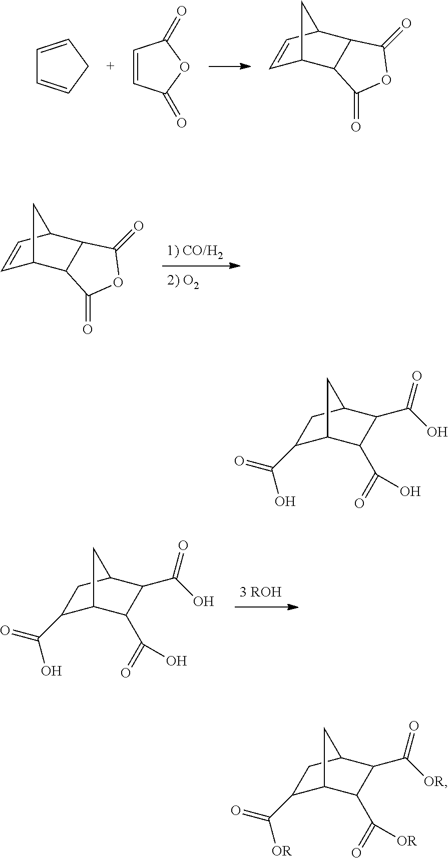

wherein ROH is a branched alcohol, preferably an OXO-alcohol, even more preferably a C₄ to C₁₄ OXO-alcohol.

In a more preferred embodiment, the resulting multi-functional compound is esterified with OXO-alcohols, which are mixed linear and branched alcohol isomers, the formation of which is described in more detail below. Esterification can be performed according to conventional processes, such as by condensation reaction of OXO-alcohol(s) with the anhydride and/or carboxylic acid portions of the molecules, either before or after the Diels-Alder reaction.

For example, when esterification is performed prior to the Diels-Alder reaction, an anhydride compound is first esterified according to the scheme below:

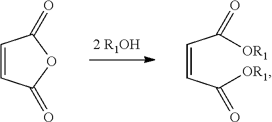

wherein R₁OH is an OXO-alcohol, followed by reacting the resulting product with cyclopentadiene via Diels-Alder reaction:

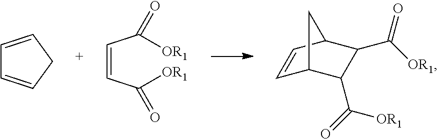

subsequently hydroformylating the carbon-carbon double bond in the presence of carbon monoxide and hydrogen to form an aldehyde of the formula:

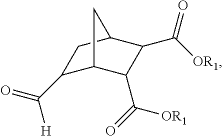

oxidizing the aldehyde to form the corresponding carboxylic acid:

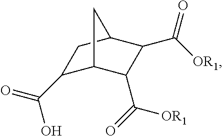

and finally esterifying the carboxylic acid moiety with the same OXO-alcohol, R₁OH, or optionally with a different OXO-alcohol, R₃OH to form a tri-ester of the formula:

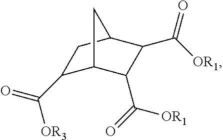

wherein each R₁ and R₃ are hydrocarbon residues of, for example C₄ to C₁₄ OXO-alcohols.

“OXO-alcohols” are isomeric mixtures of branched, organic alcohols. “OXO-esters” are compounds having at least one functional ester moiety within its structure derived from esterification of a carboxylic acid or anhydride portion or moiety of a compound with an OXO-alcohol.

OXO-alcohols can be prepared by hydroformylating olefins, followed by hydrogenation to form the alcohols. “Hydroformylating” or “hydroformylation” is the process of reacting a compound having at least one carbon-carbon double bond (an olefin) in an atmosphere of carbon monoxide and hydrogen over a cobalt or rhodium catalyst, which results in addition of at least one aldehyde moiety to the underlying compound. U.S. Pat. No. 6,482,972, which is incorporated herein by reference in its entirety, describes the hydroformylation (OXO) process. The resulting OXO-alcohols consist of multiple isomers of a given chain length due to the various isomeric olefins obtained in the oligomerization process, described below, in tandem with the multiple isomeric possibilities of the hydroformylation step.

Typically, the isomeric olefins are formed by light olefin oligomerization over heterogenous acid catalysts, such as by propylene and/or butene oligomerization over solid phosphoric acid or zeolite catalysts, which light olefins are readily available from refinery processing operations. The reaction results in mixtures of longer-chain, branched olefins, which are subsequently formed into longer chain, branched alcohols, as described below and in U.S. Pat. No. 6,274,756, incorporated herein by reference in its entirety.

Branched aldehydes are then produced by hydroformylation of the isomeric olefins. The resulting branched aldehydes can then be recovered from the crude hydroformylation product stream by fractionation to remove unreacted olefins. These branched aldehydes can then be hydrogenated to form alcohols (OXO-alcohols). Single carbon number alcohols can be used in the esterification of the anhydrides described above, or differing carbon numbers can be used to optimize product cost and performance requirements. The “OXO” technology provides cost advantaged alcohols. Other options are considered, such as hydroformylation of C₄-olefins to C₅-aldehydes, followed by hydrogenation to C₅-alcohols, or aldehyde compounding followed by hydrogenation to C₁₀-alcohols.

“Hydrogenating” or “hydrogenation” is addition of hydrogen (H₂) to a double-bonded functional site of a molecule, such as in the present case the addition of hydrogen to the aldehyde moieties of a di-aldehyde, to form the corresponding di-alcohol. Conditions for hydrogenation of an aldehyde are well known in the art and include, but are not limited to, temperatures of 0-300° C., pressures of 1-500 atmospheres, and the presence of homogeneous or heterogeneous hydrogenation catalysts such as Pt/C, Pt/Al₂O₃ or Pd/Al₂O₃.

Alternatively, the OXO-alcohols can be prepared by aldol condensation of shorter-chain aldehydes to form longer chain aldehydes, as described in U.S. Pat. No. 6,274,756, followed by hydrogenation to form the OXO-alcohols.

“Esterifying” or “esterification” is reaction of a carboxylic acid moiety, such as an anhydride, with an organic alcohol moiety to form an ester linkage. Esterification conditions are well known in the art and include, but are not limited to, temperatures of 0-300° C. and the presence or absence of homogeneous or heterogeneous esterification catalysts such as Lewis or Brønsted acid catalysts.

As discussed above, the resulting OXO-alcohols can be used individually or together in alcohol mixtures having different chain lengths, or in isomeric mixtures of the same carbon chain length to make mixed esters for use as plasticizers. This mixing of carbon numbers and/or levels of branching can be advantageous to achieve the desired compatibility with PVC for the respective core alcohol or acid used for the polar moiety end of the plasticizer, and to meet other plasticizer performance properties. The preferred OXO-alcohols are those having from 4 to 13 carbons, more preferably C₅ to C₁₀ alcohols, and even more preferably C₆ to C₇ alcohols.

In one embodiment the preferred OXO-alcohols are those which have an average branching of from 0.8 to 3.0 branches per molecule, or from 0.8 to 1.8 branches per molecule, such as between 0.8 to 1.6 branches per molecule, or between 1.1 to 1.8 branches per molecule, or 1.2 to 1.4 branches per molecule.

Typical branching characteristics of OXO-alcohols are provided in the table, below.

¹³C NMR Branching Characteristics of Typical OXO-Alcohols.

| | Avg. | % of α- | β-Branches | Total | Pendant | Pendant |
|---|---|---|---|---|---|---|
| OXO- | Carbon | Carbons w/ | per | Methyls per | Methyls per | Ethyls per |
| Alcohol | No. | Branches^{a} | Molecule^{b} | Molecule^{c} | Molecule^{d} | Molecule |
| C₄^{e} | 4.0 | 0 | 0.35 | 1.35 | 0.35 | 0 |
| C₅^{f} | 5.0 | 0 | 0.30 | 1.35 | 0.35 | 0 |
| C₆ | — | — | — | — | — | — |
| C₇ | 7.3 | 0 | 0.15 | 1.96 | 0.99 | 0.04 |
| C₈ | 8.6 | 0 | 0.09 | 3.0 | 1.5 | — |
| C₉ | 9.66 | 0 | 0.09 | 3.4 | — | — |
| C₁₀ | 10.2 | 0 | 0.16 | 3.2 | — | — |
| C₁₂ | 12.2 | 0 | — | 4.8 | — | — |
| C₁₃ | 13.1 | 0 | — | 4.4 | — | — |

— Data not available.
^{a}—COH carbon.
^{b}Branches at the—CCH₂OH carbon.
^{c}This value counts all methyl groups, including C₁ branches, chain end methyls, and methyl endgroups on C₂+ branches.
^{d}C₁ branches only.
^{e}Calculated values based on an assumed molar isomeric distribution of 65% n-butanol and 35% isobutanol (2-methylpentanol).
^{f}Calculated values based on an assumed molar isomeric distribution of 65% n-pentanol, 30% 2-methylbutanol, and 5% 3-methylbutanol.

In general, for every polymer to be plasticized, a plasticizer is required with the correct balance of solubility, volatility and viscosity to have acceptable plasticizer compatibility with the resin. In particular, if the 20° C. kinematic viscosity is higher than 200 mm²/sec as measured by the appropriate ASTM test, or alternately if the 20° C. cone-and-plate viscosity is higher than 200 cP, this will affect the plasticizer processability during formulation, and can require heating the plasticizer to ensure good transfer during storage and mixing of the polymer and the plasticizer. Volatility is also a very critical factor which affects the long-term plasticizer formulation stability. Higher volatility plasticizers can migrate from the plastic resin matrix and cause damage to the article. The plasticizer volatility in a resin matrix can be roughly predicted by neat plasticizer weight loss at 220° C. using Thermogravimetric Analysis.

One advantageous route to forming multi-ester plasticizers is to react cyclopentadiene with maleic anhydride under Diels-Alder conditions to form anhydride compounds as follows:

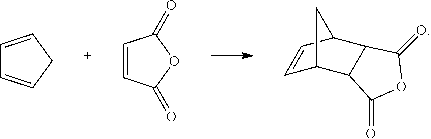 [SMILES: C1=CCC=C1 ; O=C1C=CC(=O)O1 ; O=C1OC(=O)C2C3C=CC(C3)C12]

The anhydride compounds can be hydrogenated to saturate the carbon-carbon double bond and esterified with OXO-alcohols to form diesters, as follows:

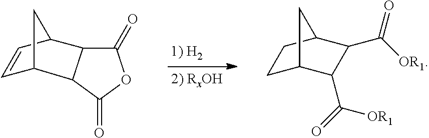

Alternatively, the anhydride compounds are reacted first with CO/H₂, followed by oxidation to form a tri-functional acid,

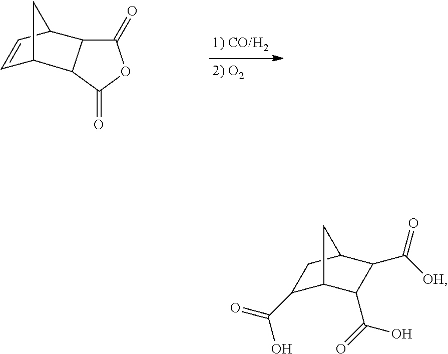 [SMILES: O=C1OC(=O)C2C3C=CC(C3)C12 ; O=C(O)C1CC2CC1C(C(=O)O)C2C(=O)O]

and subsequent esterification with OXO-alcohol(s) (RₓOH) as follows:

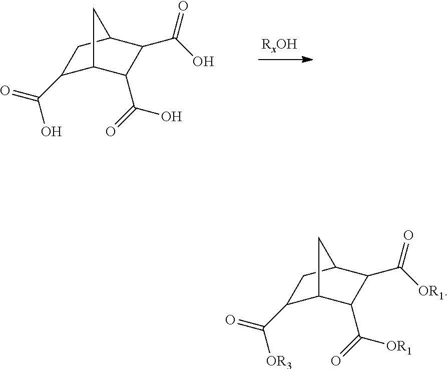

According to this reaction, R₁ and R₃ can be different if the compound is first esterified with R₁OH and subsequently reacted with CO/H₂, followed by oxidation and esterification with a different OXO-alcohol, R₃OH. This is referred to as Reppe carbonylation chemistry and is accomplished by reacting CO and an OXO-alcohol over an homogenous metal catalyst as follows:

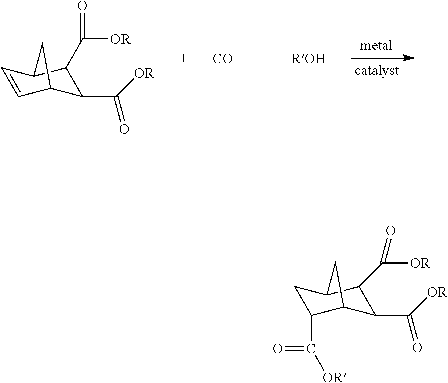

This particular method of forming the multi-esters of the present disclosure is depicted in greater detail below. Non-limiting exemplary metal catalysts for the Reppe reaction include palladium and cobalt.

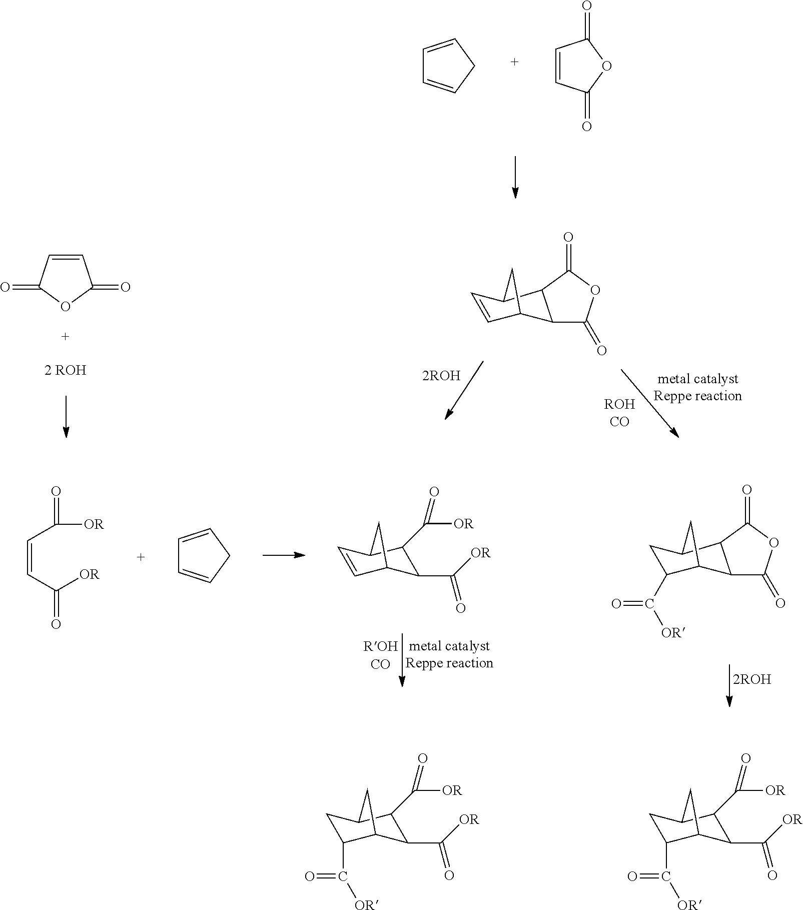

In another embodiment, the anhydride compound can be epoxidized by reacting it in the presence of a peroxy-compound under epoxidation conditions, followed by esterification with an OXO-alcohol to form epoxy-diesters, as follows:

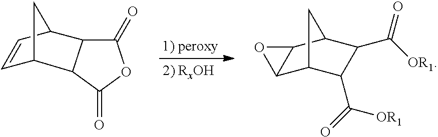

We have found that when C₄ to C₁₃ OXO-alcohols are used as reactants for the esterification reactions described above, the resulting OXO-esters are in the form of relatively high-boiling liquids (having low volatility), which are readily incorporated into polymer formulations as plasticizers.

The multi-esters disclosed herein are particularly suitable as plasticizers in polymer compositions. The plasticizers according to the current disclosure may be used with vinyl chloride-type resins, polyesters, polyurethanes, ethylene-vinyl acetate copolymer, rubbers, acrylics, polymer blends such as of polyvinyl chloride with an ethylene-vinyl acetate copolymer or polyvinyl chloride with a polyurethane or ethylene-type polymer. The multi-ester plasticizers disclosed herein are characterized as being phthalate-free. The polymer compositions including the multi-ester plasticizers disclosed herein may further include stabilizers, fillers, pigments, biocides, carbon black, adhesion promoters, viscosity reducers, thixotropic agents, thickening agents, blowing agents, and mixtures thereof. Alternatively, the polymer compositions including the multi-ester plasticizers disclosed herein may further include at least one additional plasticizer selected from phthalates, adipates, trimellitates, cyclohexanoates, benzoates, and combinations thereof.

A plastisol may also include the multi-ester plasticizers disclosed herein. An article may also be formed from the multi-ester plasticizer, or a polymer composition including the multi-ester plasticizer, or a plastisol including the multi-ester plasticizer disclosed herein. Non-limiting exemplary articles include toys, films and sheets, tubing, coated fabrics, wire and cable insulation and jacketing, flooring materials, preferably vinyl sheet flooring or vinyl floor tiles, adhesives, sealants, inks, and medical products, preferably blood bags and medical tubing. The articles may be formed by extrusion, injection molding, blow molding, and any other plastic processing technique. One preferred process includes the steps of dry blending and extrusion of a resin and the multi-ester plasticizer disclosed herein.

The following examples are meant to illustrate the present disclosure and inventive processes, and provide where appropriate, a comparison with other methods, including the products produced thereby. Numerous modifications and variations are possible and it is to be understood that within the scope of the appended claims, the disclosure can be practiced otherwise than as specifically described herein.

### EXAMPLES

### General Procedure for Esterification

Into a four necked 1000 ml round bottom flask equipped with an air stirrer, nitrogen inductor, thermometer, Dean-Stark trap and chilled water cooled condenser were added the anhydride, and the OXO-alcohol(s). The Dean-Stark trap was filled with the OXO-alcohol(s). The reaction mixture was heated to 220° C. with air stirring under a nitrogen sweep. The water collected in the Dean-Stark trap was drained frequently. The theoretical weight of water was obtained in 3 hours at 220° C. indicating 96% conversion. The reaction mixture was heated longer to achieve complete conversion to the diester. Excess alcohols plus some mono esters were removed by distillation. The crude residual product was treated with decolorizing charcoal with stirring at room temperature overnight. The mixture was then filtered twice to remove the charcoal.

### Example 1

### Esterification of cis-1,2-cyclohexanedicarboxylic acid with OXO—C₉ Alcohols

Into a 4-necked 1000 ml round bottom flask equipped with an air stirrer, thermometer, N2 inductor, Dean-Stark trap and chilled water cooled condenser were added cis-1,2-cyclohexanedicarboxylic acid (172.18 g, 1.0 mole), OXO—C₉ alcohols (433.2 g, 3.0 mole) and OXO—C₉ alcohols (16 g, 0.11 mole) were added to the Dean-Stark trap. The reaction mixture was heated at total of 5.5 hours at 157-220° C. with gas chromatographic (GC) sampling. A total of 31 grams water was drained from the Dean-Stark trap which corresponds to 94% of theoretical. The product was concentrated using a Claisen adapter, chilled water cooled condenser and receiving flask. The crude product was a hazy colorless liquid. After treatment with decolorizing charcoal at room temperature with stirring for 2 hours and filtration, a clear and colorless liquid was obtained which was diisononyl cyclohexanoate (DINCH) with a purity of 99.93% by GC.

### Example 2

### Esterification of 5-norbornane-2,3-dicarboxylic acid with OXO—C₉ Alcohols

Into a 4-necked 500 ml round bottom flask equipped with an air stirrer, thermometer, N2 inductor, Dean-Stark trap and chilled water cooled condenser were added 5-norbornane-2,3-dicarboxylic acid (101.5 g, 0.551 mole), OXO—C₉ alcohols (238.7 g, 1.65 mole) and OXO—C₉ alcohols (15.5 g, 0.11 mole) were added to the Dean-Stark trap. The reaction mixture was heated at total of 6 hours at 150-224° C. with GC sampling. A total of 18.8 grams water was drained from the Dean-Stark trap which corresponds to 95% of theoretical. The product was concentrated using a Claisen adapter, chilled water cooled condenser and receiving flask. The crude product was treated with decolorizing charcoal at room temperature with stirring for 12 hours then filtered twice, a clear and colorless liquid was obtained which was diisononylbicyclo[2.2.1]heptane-2,3-dicarboxylate (DINCH+1) with a purity of 99.22% by GC.

### Example 3

### Epoxidation Procedures

In a one liter round bottom flask equipped with a stir bar, thermometer and graduated addition funnel was added 25 g (0.12 mole) of dimethyl-5-norbornene-2,3-dicarboxylate and 175 ml methylene chloride. The addition funnel was charged with g (0.13 mole) of 77% 3-chloroperoxybenzoic acid and 250 ml methylene chloride then added slowly over 15 minutes maintaining the temperature at 20-25° C. using a cold water bath. After addition the mixture was stirred for one hour and a sample was taken to determine reaction completeness by GC-MS. The reaction mixture was treated with a 10% sodium sulfite solution to destroy any excess peracid. The mixture was treated with 5% sodium bicarbonate to remove excess 3-chlorobenzoic acid followed by washing with saturated sodium chloride. The solution was dried over magnesium sulfate filtered and the methylene chloride was removed under vacuum to give epoxy diisononylbicyclo[2.2.1]heptane-2,3-dicarboxylate (epoxy DINCH+1).

### Example 4

### Transesterification of Dimethyl-5,6-epoxynorbornane-2,3-dicarboxylate with OXO—C₉ Alcohols

Into a 4-necked 250 ml round bottom flask equipped with an air stirrer, N₂ inductor, condenser, in, out and by-pass nitrogen bubblers were added the dimethyl-5,6-epoxynorbornane-2,3-dicarboxylate (17.3 g, 0.077 mole) and OXO—C₉ alcohols (44.2 g, 0.3061 mole). The reaction mixture was heated at 150° C. with air stirring for 12 hours with GC sampling to monitor reaction. The product formed was epoxy diisononylbicyclo[2.2.1]heptane-2,3-dicarboxylate (Epoxy DINCH+1).

### General Procedure for the Use of Esters to Plasticize Poly(Vinyl Chloride)

A 4.5 g portion of the ester sample was weighed into an Erlenmeyer flask which had previously been rinsed with uninhibited tetrahydrofuran (THF) to remove dust. A 0.63 g portion of a 70:30 by weight solid mixture of powdered Drapex® 6.8 (Crompton Corp.) and Mark® 4716 (Chemtura USA Corp.) stabilizers were added along with a stirbar. The solids were dissolved in 90 mL uninhibited THF. Oxy Vinyls® 240F PVC (9.0 g) was added in powdered form and the contents of the flask were stirred overnight at room temperature until dissolution of the PVC was complete (a PVC solution for preparation of an unplasticized comparative sample was prepared using an identical amount of stabilizer, 100 mL solvent, and 13.5 g PVC). The clear solution was poured evenly into a flat aluminum paint can lid (previously rinsed with inhibitor-free THF to remove dust) of dimensions 7.5″ diameter and 0.5″ depth. The lid was placed into an oven at 60° C. for 2 hours with a moderate nitrogen purge. The pan was removed from the oven and allowed to cool for a ˜5 min period. The resultant clear film was carefully peeled off of the aluminum, flipped over, and placed back evenly into the pan. The pan was then placed in a vacuum oven at 70° C. overnight to remove residual THF. The dry, flexible, almost colorless film was carefully peeled away and exhibited no oiliness or inhomogeneity. The film was cut into small pieces to be used for preparation of test bars by compression molding (size of pieces was similar to the hole dimensions of the mold plate). The film pieces were stacked into the holes of a multi-hole steel mold plate, pre heated to 170° C., having hole dimensions 20 mm×12.8 mm×1.8 mm (ASTM D1693-95 dimensions). The mold plate was pressed in a PHI Company QL-433-6-M2 model hydraulic press equipped with separate heating and cooling platforms. The upper and lower press plates were covered in Teflon™-coated aluminum foil and the following multistage press procedure was used at 170° C. with no release between stages: (1) 3 minutes with 1-2 ton overpressure; (2) 1 minute at 10 tons; (3) 1 minute at 20 tons; (4) 1 minute at 30 tons; (5) 3 additional minutes at 30 tons; (6) release and 3 minutes in the cooling stage of the press (7° C.) at 30 tons. A knockout tool was then used to remove the sample bars with minimal flexion. Near-colorless, flexible bars were obtained which, when stored at room temperature, showed no oiliness or exudation several weeks after pressing. The bars were allowed to age at room temperature for at least 1 week prior to evaluation of phase behavior with Differential Scanning Calorimetry (DSC) and thermo-physical properties with Dynamic Mechanical Analysis (DMA).

### Differential Scanning Calorimetry (DSC) and Thermogravimetric Analysis (TGA) Property Study of Esters and Plasticized Bars

Thermogravimetric Analysis (TGA) was conducted on the neat esters and compression-molded PVC plasticized bars prepared above (PVC:plasticizer ratio=2:1) using a TA Instruments TGA5000 instrument (25-500° C., 10° C./min, under 25 cc N₂/min flow through furnace and 10 cc N₂/min flow through balance; sample size approximately mg). Table 1 provides a volatility comparison of the exemplary ester fractions and diisononyl phthalate (DINP), along with a comparison of the glass transitions (T_{g}) of the different ester fractions. Table 2 provides a volatility comparison of the neat and plasticized PVC bars as measured by percentage weight loss at varying temperatures. Differential Scanning Calorimetry (DSC) was also performed on the neat plasticizers (Table 1), using a TA Instruments Q2000 calorimeter fitted with a liquid N₂ cooling accessory. Samples were loaded at room temperature and cycled at 10° C./min between −130° C. and 50° C. T_{g} is given in Table 1 are calculated as the half-height of the step-change in heat capacity during the second heat (unless only one heat cycle was performed, in which case the first heat T_{g} is given, which is typically in very close agreement).

TABLE 1
Volatility and Glass Transition Properties of Esters.

| | | | TGA | TGA | DSC |
|---|---|---|---|---|---|
| | TGA 1% Wt | TGA 5% Wt | 10% Wt | Wt Loss at | Tg |
| Sample | Loss (° C.) | Loss (° C.) | Loss (° C.) | 220° C. (%) | (° C.) |
| DINP | 184.6 | 215.2 | 228.5 | 6.4 | −79.1 |
| Ex. 1 | 180.4 | 212.5 | 226 | 7.2 | −90.5 |
| Ex. 2 | 180 | 214.3 | 229.6 | 6.5 | −77.8 |

TABLE 2
Volatility Properties of Neat PVC and
Plasticized PVC Sample Bars.

| Plasticizer | | | TGA | TGA |
|---|---|---|---|---|
| Used | TGA 1% Wt | TGA 5% Wt | 10% Wt | % Wt Loss |
| in Bar | Loss (° C.) | Loss (° C.) | Loss (° C.) | at 220° C. |
| None | 129.9 | 192.3 | 255.4 | 6.3 |
| (Neat PVC) | | | | |
| DINP | 194.9 | 240.9 | 253.7 | 2.4 |
| Ex. 1 | 185.7 | 230.0 | 249.8 | 3.5 |
| Ex. 2 | 193.0 | 237.0 | 251.8 | 2.7 |

Table 1 demonstrates that DINCH+1 (Ex.2) behaves similarly to DINP. Neat ester volatility is improved over DINCH (Ex.1). Table 2 confirms improved volatility of DINCH+1 over DINCH in plasticized PVC bars.
Demonstration of Plasticization of PVC with Different Esters Made Using this Disclosure Via Differential Scanning Calorimetry (DSC)

Differential Scanning Calorimetry (DSC) was performed on the compression-molded sample bars prepared above (PVC:plasticizer ratio=2:1) using a TA Instruments Q2000 calorimeter. Small portions of the sample bars (typical sample mass 4-7 mg) were cut for analysis, making only vertical cuts perpendicular to the largest surface of the bar to preserve the upper and lower compression molding “skins”. Samples were loaded at room temperature and cycled at 10° C./min between approximately −110 and 100° C. Table 3 provides the first heat T_{g} onset, T_{g} midpoint, and T_{g} end for neat PVC and the plasticized bars.

TABLE 3
Glass Transition Onset, Midpoint, and
End for Plasticized PVC Bars

| | | | | Tm Max |
|---|---|---|---|---|
| | | | | (° C.) |
| Plasticizer Used in | Tg Onset | Tg Midpt | Tg End | and |
| Bar | (° C.) | (° C.) | (° C.) | DH_{f} (J/g) |
| None (Neat PVC) | −56.1 | −40.6 | −25 | not calc. |
| DINP | −45.7 | −22.98 | −0.34 | 54.4 (0.5) |
| Ex. 1 | −58.7 | −33.4 | −8.1 | 54.5 (0.4) |
| Ex. 2 | −55.1 | −32.5 | −10.0 | 53.6 (0.6) |

A lowering and broadening of the glass transition for neat PVC is observed upon addition of the esters, indicating plasticization and extension of the flexible temperature range of use for neat PVC.

Demonstration of Plasticization of PVC with Different Esters Via Dynamic Mechanical Analysis (DMA)

A TA Instruments DMA Q800 fitted with a liquid N₂ cooling accessory and a three-point bend clamp assembly was used to measure the thermo-mechanical performance of neat PVC and the PVC/plasticizer blend sample bars prepared above. Samples were loaded at room temperature and cooled to −90° C. at a cooling rate of 3° C./min. After equilibration, a dynamic experiment was performed at one frequency using the following conditions: 3° C./min heating rate, 1 Hz frequency, 20 μm amplitude, 0.01 N pre-load force, force track 120%. Two or three bars of each sample were typically analyzed and numerical data was averaged. The DMA measurement gives storage modulus (elastic response modulus) and loss modulus (viscous response modulus); the ratio of loss to storage moduli at a given temperature is tan delta. The beginning (onset) of the T, (temperature of brittle-ductile transition) was obtained for each sample by extrapolating a tangent from the steep inflection of the tan delta curve and the first deviation of linearity from the baseline prior to the beginning of the peak. Table 4 provides a number of DMA parameters for neat PVC and PVC bars plasticized with the esters: T_{g} onset (taken from tan delta); peak of the tan delta curve; storage modulus at 25° C.; and the temperature at which the storage modulus equals 100 MPa (this temperature was chosen to provide a measure of the temperature at which the PVC loses a set amount of rigidity, too much loss of rigidity may lead to processing complications for the PVC material.). The flexible use temperature range of the plasticized PVC samples is evaluated as the range between the T_{g} onset and the temperature at which the storage modulus was 100 MPa.

TABLE 4
Various DMA Thermal Parameters for Plasticized PVC Bars

| | | | | Temp. of | |
|---|---|---|---|---|---|
| | Tan δ | | 25° C. | 100 MPa | Flexible |
| Plasticizer | T_{g} | Tan δ | Storage | Storage | Use |
| Used in | Onset | Peak | Mod. | Mod. | Range |
| Bar | (° C.) | (° C.) | (MPa) | (° C.) | (° C.) |
| None | 44.0 | 61.1 | 1433 | 57.1 | 13.1 |
| (Neat | | | | | |
| PVC) | | | | | |
| DINP | −38.0 | 10.0 | 35.7 | 13.1 | 51.1 |
| Ex. 1 | −40.4 | 11.7 | 47.8 | 12.9 | 53.2 |
| Ex. 2 | −42.7 | 36.2 | 86.0 | 23.1 | 65.8 |

A lowering and broadening of the glass transition for neat PVC is observed upon addition of the esters, indicating plasticization and extension of the flexible temperature range of use for neat PVC. DINCH+1 is particularly effective resulting in the lowest tan δ onset and largest flexible use range. Plasticization (enhanced flexibility) is also demonstrated by lowering of the PVC room temperature storage modulus upon addition of the esters.

TABLE 5
Summary of plasticizing performance

| | | | | Weight loss | | Notes on |
|---|---|---|---|---|---|---|
| | | | Viscosity | @ 220° C. | | films or |
| Sample # | alcohol | di-acid | (cP) | (wt %) | Tg | bars |
| DINP | OXO- | Phthalic anhydride | 96.81 | 6.4 | −79.1 | OK |
| | C₉ | | | | | |
| Ex. 2 | OXO- | 5-norbornane-2,3- | 98.47 | 6.5 | −77.8 | Little |
| | C₉ | dicarboxylic acid | | | | stiffer than |
| | | | | | | DINP |

Table 5 shows that the viscosity, weight lost at 220° C. and Tg of DINCH+1 is similar to DINP, thus the two plasticizers will behave similarly in plasticizing PVC.

The meanings of terms used herein shall take their ordinary meaning in the art; reference shall be taken, in particular, to Handbook of Petroleum Refining Processes, Third Edition, Robert A. Meyers, Editor, McGraw-Hill (2004). In addition, all patents and patent applications, test procedures (such as ASTM methods), and other documents cited herein are fully incorporated by reference to the extent such disclosure is not inconsistent with this disclosure and for all jurisdictions in which such incorporation is permitted. Also, when numerical lower limits and numerical upper limits are listed herein, ranges from any lower limit to any upper limit are contemplated. Note further that Trade Names used herein are indicated by a ™ symbol or ® symbol, indicating that the names may be protected by certain trademark rights, e.g., they may be registered trademarks in various jurisdictions.

Applicants have attempted to disclose all embodiments and applications of the disclosed subject matter that could be reasonably foreseen. However, there may be unforeseeable, insubstantial modifications that remain as equivalents. While the present disclosure has been described in conjunction with specific, exemplary embodiments thereof, it is evident that many alterations, modifications, and variations will be apparent to those skilled in the art in light of the foregoing description without departing from the spirit or scope of the present disclosure. Accordingly, the present disclosure is intended to embrace all such alterations, modifications, and variations of the above detailed description.

All patents, test procedures, and other documents cited herein, including priority documents, are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated.

What is claimed is:

1. Multi-esters of the formula: 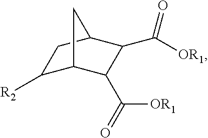
wherein each R₁ is the hydrocarbon residue of a C₄ to C₁₃ OXO-alcohol averaging from 0.2 to 5.0 branches per residue, and R₂ is hydrogen or an ester group.

2. The multi-esters of claim 1, wherein the hydrocarbon residue averages from 0.05 to 0.4 branches per residue at the alcoholic beta carbon.

3. The multi-esters of claim 1, wherein the hydrocarbon residue averages at least 1.3 to 5.0 methyl branches per residue.

4. The multi-esters of claim 1, wherein the hydrocarbon residue averages from 0.35 to 1.5 pendant methyl branches per residue.

5. The multi-esters of claim 1, wherein R₂ is H and the multi-esters are diesters.

6. The multi-esters of claim 1, which are tri-esters of the formula: 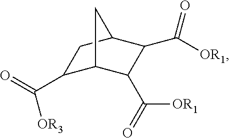
wherein R₁ and R₃ are hydrocarbon residues of the same or different OXO-alcohols.

7. The multi-esters of claim 6, wherein R₁ and R₃ are hydrocarbon residues of the same OXO-alcohol, having at least one pendant methyl branch per residue.

8. A process for making multi-esters comprising:
reacting cyclopentadiene with maleic anhydride to form compounds of the following formula (I): 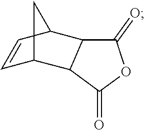 [SMILES: O=C1OC(=O)C2C3C=CC(C3)C12]
optionally conducting a first esterification step by contacting said compounds of formula (I) with at least one C₄ to C₁₃ OXO-alcohol of the formula R₁OH under esterification conditions to form compounds of the following formula (II): 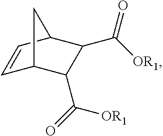
wherein R₁ is a hydrocarbon residue of the OXO-alcohol R₁OH;
hydroformylating said compounds of formulae (I) or (II), and oxidizing said hydroformylated compounds to form multi-functional compounds of the following formula (IIa): 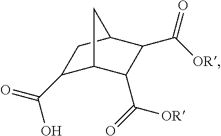
wherein R′ can be H or R₁; and
reacting said multi-functional compounds of formula (IIa) with OXO-alcohols of the formula R₁OH if R′ is H, or if R′ is R₁ with an OXO-alcohol of the formula R₃OH, under esterification conditions to form tri-esters of the following formula: 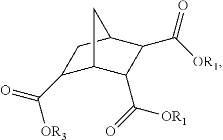
wherein each R₁ and R₃ are the same or different hydrocarbon residues of said C₄ to C₁₃ OXO-alcohols averaging from 0.2 to 5.0 branches per residue.

9. The process of claim 8, wherein the hydrocarbon residue averages from 0.05 to 0.4 branches per residue at the alcoholic beta carbon.

10. The process of claim 8, wherein the hydrocarbon residue averages at least 1.3 to 5.0 methyl branches per residue.

11. The process of claim 8, wherein the hydrocarbon residue averages from 0.35 to 1.5 pendant methyl branch per residue.

12. The process of claim 8, wherein each R₁ and R₃ are the hydrocarbon residue of the same OXO-alcohol, having at least one pendant methyl branch per residue.

13. A process for making multi-esters comprising:
contacting maleic anhydride with at least one C₄ to C₁₃ OXO-alcohol of the formula R₁OH under esterification conditions to form compounds of the following formula (III): 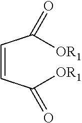
reacting the compounds of formula (III) with cyclopentadiene to form compounds of the following formula (II): 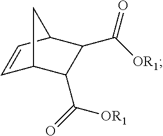
hydroformylating said compounds of formula (II), and oxidizing said hydroformylated compounds to form multi-functional compounds of the following formula (IIb): 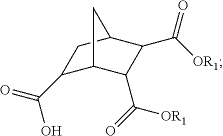
and
reacting said multi-functional compounds of formula (IIb) with an OXO-alcohol of the formula R₃OH, under esterification conditions to form tri-esters of the following formula: 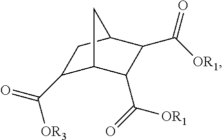
wherein each R₁ and R₃ are the same or different hydrocarbon residues of said C₄ to C₁₃ OXO-alcohols averaging from 0.2 to 5.0 branches per residue.

14. The process of claim 13, wherein the hydrocarbon residue averages from 0.05 to 0.4 branches per residue at the alcoholic beta carbon.

15. The process of claim 13, wherein the hydrocarbon residue averages at least 1.3 to 5.0 methyl branches per residue.

16. The process of claim 13, wherein the hydrocarbon residue averages from 0.35 to 1.5 pendant methyl branch per residue.

17. The process of claim 13, wherein each R₁ and R₃ are the hydrocarbon residues of the same OXO-alcohol, having at least one pendant methyl branch per residue.

18. A process for making multi-esters comprising:
reacting cyclopentadiene with maleic anhydride to form compounds of the following formula (I): 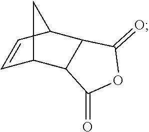 [SMILES: O=C1OC(=O)C2C3C=CC(C3)C12]
reacting the compounds of formula (I) with CO and OXO-alcohols of the formula R₁OH if R′ is H, or if R′ is R with an OXO-alcohol of the formula R₃OH, in the presence of a metal catalyst to form compounds of the following formula (IV): 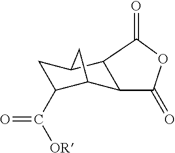
reacting the compounds of formula (IV) with OXO-alcohols of the formula R₁OH if R′ is H, or if R′ is R₁ with an OXO-alcohol of the formula R₃OH, under esterification conditions to form tri-esters of the following formula: 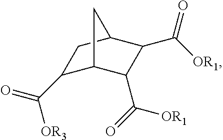
wherein each R₁ and R₃ are the same or different hydrocarbon residues of said C₄ to C₁₃ OXO-alcohols averaging from 0.2 to 5.0 branches per residue.

19. The process of claim 18, wherein the hydrocarbon residue averages from 0.05 to 0.4 branches per residue at the alcoholic beta carbon.

20. The process of claim 18, wherein the hydrocarbon residue averages at least 1.3 to 5.0 methyl branches per residue.

21. The process of claim 18, wherein the hydrocarbon residue averages from 0.35 to 1.5 pendant methyl branch per residue.

22. The process of claim 18, wherein each R₁ and R₃ are the hydrocarbon residue of the same OXO-alcohol, having at least one pendant methyl branch per residue.

23. A process for making multi-esters comprising:
reacting cyclopentadiene with maleic anhydride to form compounds of the following formula (I): 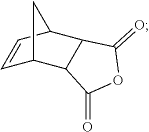 [SMILES: O=C1OC(=O)C2C3C=CC(C3)C12]
conducting a first esterification step by contacting said compounds of formula (I) with at least one C₄ to C₁₃ OXO-alcohol of the formula R₁OH under esterification conditions to form compounds of the following formula (II): 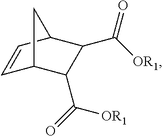
wherein R₁ is a hydrocarbon residue of the OXO-alcohol R₁OH;
reacting the compounds of formula (II) with CO and OXO-alcohols of the formula R₁OH if R′ is H, or if R′ is R₁ with an OXO-alcohol of the formula R₃OH, in the presence of a metal catalyst to form tri-esters of the following formula: 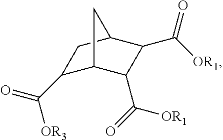
wherein each R₁ and R₃ are the same or different hydrocarbon residues of said C₄ to C₁₃ OXO-alcohols averaging from 0.2 to 5.0 branches per residue.

24. The process of claim 23, wherein the hydrocarbon residue averages from 0.05 to 0.4 branches per residue at the alcoholic beta carbon.

25. The process of claim 23, wherein the hydrocarbon residue averages at least 1.3 to 5.0 methyl branches per residue.

26. The process of claim 23, wherein the hydrocarbon residue averages from 0.35 to 1.5 pendant methyl branch per residue.

27. The process of claim 23, wherein each R₁ and R₃ are the hydrocarbon residue of the same OXO-alcohol, having at least one pendant methyl branch per residue.

28. A process for making multi-esters comprising:
contacting maleic anhydride with at least one C₄ to C₁₃ OXO-alcohol of the formula R₁OH under esterification conditions to form compounds of the following formula (III): 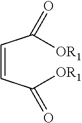
reacting the compounds of formula (III) with cyclopentadiene to form compounds of the following formula (II): 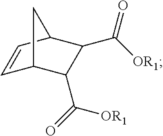
reacting the compounds of formula (II) with CO and OXO-alcohols of the formula R₁OH if R′ is H, or if R′ is R₁ with an OXO-alcohol of the formula R₃OH, in the presence of a metal catalyst to form tri-esters of the following formula: 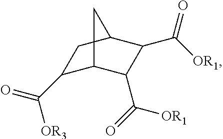
wherein each R₁ and R₃ are the same or different hydrocarbon residues of said C₄ to C₁₃ OXO-alcohols averaging from 0.2 to 5.0 branches per residue.

29. The process of claim 28, wherein the hydrocarbon residue averages from 0.05 to 0.4 branches per residue at the alcoholic beta carbon.

30. The process of claim 28, wherein the hydrocarbon residue averages at least 1.3 to 5.0 methyl branches per residue.

31. The process of claim 28, wherein the hydrocarbon residue averages from 0.35 to 1.5 pendant methyl branch per residue.

32. The process of claim 28, wherein each R₁ and R₃ are the hydrocarbon residue of the same OXO-alcohol, having at least one pendant methyl branch per residue.

33. A plasticizer comprising the multi-esters of the formula: 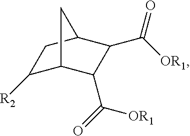
wherein each R₁ is the hydrocarbon residue of a C₄ to C₁₃ OXO-alcohol averaging from 0.2 to 5.0 branches per residue, and R₂ is hydrogen or an ester group.

34. The plasticizer of claim 33 characterized as being phthalate-free.

35. A polymer composition comprising a polymer and at least one plasticizer comprising multi-esters of the formula: 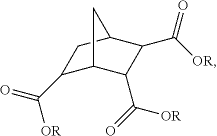
wherein each R is the hydrocarbon residue of a C₄ to C₁₃ OXO-alcohol averaging from 0.2 to 5.0 branches per residue.

36. The polymer composition of claim 35, wherein the polymer is selected from the group consisting of polyvinylchlorides, polyesters, polyurethanes, ethylene-vinyl acetate copolymers, rubbers, poly(meth)acrylics and combinations thereof.

37. The polymer composition of claim 35, further comprising stabilizers, fillers, pigments, biocides, carbon black, adhesion promoters, viscosity reducers, thixotropic agents, thickening agents, blowing agents, and mixtures thereof.

38. The polymer composition of claim 35, further comprising at least one plasticizer selected from phthalates, adipates, trimellitates, cyclohexanoates, benzoates, and combinations thereof.

39. A plastisol comprising the plasticizer of claim 33.

40. An article comprising the plasticizer of claim 33, the polymer composition of claim 35, or the plastisol of claim 39.

41. The article of claim 40, wherein the article is selected from toys, films and sheets, tubing, coated fabrics, wire and cable insulation and jacketing, flooring materials, preferably vinyl sheet flooring or vinyl floor tiles, adhesives, sealants, inks, and medical products, preferably blood bags and medical tubing.

42. The article of claim 41, made by a process including steps of dry blending and extrusion.
